(19) United States
(12) Patent Application Publication
(10) Pub. No.: US 2023/0115023 A1
(43) Pub. Date: Apr. 13, 2023

(54) USE OF EXOSOMES DERIVED FROM MESENCHYMAL STEM CELLS FORTREATING NON-ALCOHOLIC STEATOHEPATITIS

(71) Applicant: CHINA MEDICAL UNIVERSITY, Taichung (TW)

(72) Inventors: Oscar Kuang-Sheng LEE, Taichung (TW); Chien-Wei LEE, Taichung (TW); Yu-Fan CHEN, Taichung (TW); Hao-Hsiang WU, Taichung (TW)

(21) Appl. No.: 17/938,607

(22) Filed: Oct. 6, 2022

Related U.S. Application Data
(60) Provisional application No. 63/253,484, filed on Oct. 7, 2021.

(51) Int. Cl. A61K 35/28; C12N 5/0775; A61P 1/16

(52) U.S. Cl. CPC A61K 35/28; C12N 5/0665; A61P 1/16

(57) ABSTRACT

The present disclosure provides a method for treating non-alcoholic steatohepatitis and/or non-alcoholic fatty liver disease in a subject in need thereof, the method comprises administering exosome, an exosome pellet, or physiological solution derived from mesenchymal stem cells (MSCs) to the subject.

### PRIORITY INFORMATION

The present application claims priority to and benefit of U.S. Provisional Patent Application No. 63/253,484, filed Oct. 7, 2021, the disclosure of which is incorporated in its entirety herein.

### FIELD OF THE INVENTION

The present invention relates to a method for treating non-alcoholic fatty liver disease. Particularly, a method for treating non-alcoholic fatty liver disease using exosomes derived from stem cells.

### BACKGROUND OF THE INVENTION

Non-alcoholic steatohepatitis (NASH), a common chronic liver disease, manifests as a fatty inflammation of the liver and is a major cause of cirrhosis, fibrosis, and liver failure. The disease is progressive, starting as steatosis or nonalcoholic fatty liver disease (NAFLD), progressing to an inflamed fatty liver (NASH), and eventually leading to cirrhosis and fibrosis. The disease is generally asymptomatic until severe liver impairment occurs. Patients having NASH are also often characterized by abnormal levels of liver enzymes, such as aspartate aminotransferase (AST) and alanine aminotransferase (ALT) due to lipid accumulation caused lipotoxicity. Currently, there are few therapies to slow or alter the course of further disease progression in NASH.

Therefore, there remains a need for effective NASH treatment.

### SUMMARY OF THE INVENTION

The present disclosure provides a method for treating non-alcoholic steatohepatitis in a subject in need thereof, wherein the method comprises administering an exosome, exosome pellet, or physiological solution derived from mesenchymal stem cells (MSCs) to the subject.

The present disclosure provides a method for treating non-alcoholic fatty liver disease in a subject in need thereof, wherein the method comprises administering an exosome, exosome pellet, or physiological solution derived from mesenchymal stem cells to the subject.

The present disclosure provides a method for stimulating serum alanine aminotransferase expression in a subject in need thereof, wherein the method comprises administering an exosome, exosome pellet, or physiological solution derived from mesenchymal stem cells to the subject. Alternatively, the present disclosure provides a pharmaceutical composition for stimulating serum alanine aminotransferase expression in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for stimulating serum alanine aminotransferase expression in a subject in need thereof.

The present disclosure provides a method for reducing controlled attenuation parameter of liver fat in a subject in need thereof, wherein the method comprises administering an exosome, exosome pellet, or physiological solution derived from mesenchymal stem cells to the subject. Alternatively, the present disclosure provides a pharmaceutical composition for reducing controlled attenuation parameter of liver fat in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for reducing controlled attenuation parameter of liver fat in a subject in need thereof.

The present disclosure provides a method for reducing hepatic lipid accumulation in a subject in need thereof, wherein the method comprises administering an exosome, exosome pellet, or physiological solution derived from mesenchymal stem cells to the subject. Alternatively, the present disclosure provides a pharmaceutical composition for reducing hepatic lipid accumulation in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for reducing hepatic lipid accumulation in a subject in need thereof.

The present disclosure provides a method for decreasing hepatic lipotoxicity in a subject in need thereof, wherein the method comprises administering an exosome, exosome pellet, or physiological solution derived from mesenchymal stem cells to the subject. Alternatively, the present disclosure provides a pharmaceutical composition for decreasing hepatic lipotoxicity in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for decreasing hepatic lipotoxicity in a subject in need thereof.

The present disclosure provides a method for ameliorating PPAR dysregulation in the liver in a subject in need thereof, wherein the method comprises administering an exosome, exosome pellet, or physiological solution derived from mesenchymal stem cells to the subject. In some embodiments of the disclosure, the method is for decreasing PPARα and/or PPARγ expression. Alternatively, the present disclosure provides a pharmaceutical composition for ameliorating PPAR dysregulation in the liver in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for ameliorating PPAR dysregulation in the liver in a subject in need thereof.

The present disclosure provides a method for reducing body weight in a subject in need thereof, wherein the method comprises administering an exosome, exosome pellet, or physiological solution derived from mesenchymal stem cells to the subject. In some embodiments of the disclosure, the subject receives high-fat diet. Alternatively, the present disclosure provides a pharmaceutical composition for reducing body weight in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for reducing body weight in a subject in need thereof.

The present disclosure provides a method for reducing glycated hematocrit in a subject in need thereof, wherein the method comprises administering an exosome, exosome pellet, or physiological solution derived from mesenchymal stem cells to the subject. Alternatively, the present disclosure provides a pharmaceutical composition for reducing glycated hematocrit in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for reducing glycated hematocrit in a subject in need thereof.

The present disclosure provides a method for improving insulin resistance in a subject in need thereof, wherein the method comprises administering an exosome, exosome pellet, or physiological solution derived from mesenchymal stem cells to the subject. Alternatively, the present disclosure provides a pharmaceutical composition for improving insulin resistance in a subject in need thereof, comprising an exosome, exosome pellet, or physiological solution derived from mesenchymal stem cells. Alternatively, the present disclosure provides a use of an exosome, exosome pellet, or physiological solution derived from mesenchymal stem cells for the manufacture of a medicament for improving insulin resistance in a subject in need thereof.

In some embodiments of the disclosure, the NAFLD is secondary NAFLD, steatosis, progressive fibrosis, liver failure or cirrhosis.

In some embodiments of the disclosure, the secondary NAFLD is an NAFLD resulted from the use of one or more of amiodarone, antiviral drugs such as nucleoside analogues, aspirin or NSAIDs, corticosteroids, methotrexate, nifedipine, perhexyline, tamoxifen, tetracycline, or 5 valproic acid.

In some embodiments of the disclosure, the exosome, exosome pellet, or physiological solution is administered by injection.

In some embodiments of the disclosure, the exosome, exosome pellet, or physiological solution is administered more than one times.

In some embodiments of the disclosure, the exosome, exosome pellet, or physiological solution is administered every other day.

In some embodiments of the disclosure, the exosome, exosome pellet, or physiological solution described herein is obtained by culturing cells in a medium for a period of time sufficient to obtain an amount of cells ranging from about 1×10⁵ cells/mL to about 1×10¹⁰ cells/mL.

In some embodiments of the disclosure, the MSCs are umbilical cord mesenchymal stem cells (UMSCs), adipose derived mesenchymal stem cells (ADSCs), or bone marrow mesenchymal stem cells (BMSCs).

In some embodiments of the disclosure, the exosome, an exosome pellet, or physiological solution is provided in a pharmaceutical composition in an effective amount for treating the non-alcoholic steatohepatitis or non-alcoholic fatty liver disease, reducing controlled attenuation parameter of liver fat, reducing hepatic lipid accumulation, reducing glycated hematocrit, or improving insulin resistance.

Certain embodiments of the effective amount of the exosomes of the present disclosure are those ranging from about 1×10⁵ particles to about 1×10¹⁵ particles. In some embodiments, the effective amount of the exosomes of the present disclosure are those ranging from about 1×10⁶ particles to about 1×10¹⁴ particles, from about 1×10⁷ particles to about 1×10¹³ particles, from about 1×10⁸ particles to about 1×10¹² particles, from about 1×10⁸ particles to about 1×10¹¹ particles, from about 1×10⁸ particles to about 1×10¹⁰ particles, from about 5×10⁸ particles to about 5×10⁹ particles, about 1×10⁹ particles.

### BRIEF DESCRIPTION OF THE DRAWINGS

FIGS. 1A and 1B show that exosomes from MSCs decrease lipid accumulation in hepatoblast cell line, FL83B cells. FL83B was pretreated with free fatty acids 200 μM (palmitic acid to oleic acid with 2:1) for 24 hours and then treated with exosomes from MSCs (10 μg per 6-well) for one day. Nile red staining revealed lipid accumulation in FL83B. FIG. 1A shows that representative images and quantitation of Oil red O staining revealed lipid accumulation in FL83B. FIG. 1B shows that cell number revealed the cell survival of FL83B.

FIGS. 2A and 2B show that exosomes from human MSCs decrease fatty acid-induced intracellular lipid accumulation and lipotoxicity in a hepatoblast cell line, FL83B cells. FIG. 2A shows that representative images and quantitation of Oil red O staining revealed lipid accumulation in FL83B. FIG. 2B shows that cell number revealed the cell survival of FL83B. Statistical analyses were performed using one-way ANOVA with Tukey's multiple comparison test, with significance set at P<0.05. (*P<0.05; **P<0.01; ***P<0.001; ****P<0.0001).

FIG. 3 shows that exosomes from human MSCs ameliorate PPAR dysregulation in a hepatoblast cell line, FL83B cells. FL83B cells were exposed to LPO medium to mimic hepatic steatosis and treated with or without exosomes from MSCs. Real-time qPCR revealed the expression of PPARα and PPARγ, the master regulators of lipid metabolism, in FL83B cells. LPO medium, DMEM/F12 medium+10% FBS+1% PSG+10 mM L-lactate+1 mM sodium pyruvate+4 mM octanoic acid.

FIGS. 4A to 4C shows that exosome from human MSCs reduced body weight in HFD mice. FIG. 4A shows experimental setup. FIG. 4B shows that HFD increased body weight in mice. FIG. 4C shows that exosome injection (10⁹ particle per injection) reduced body weight in HFD mice. High-fat diet, HFD.

### DETAILED DESCRIPTION OF THE INVENTION

Unless defined otherwise, all scientific or technical terms used herein have the same meaning as those understood by persons of ordinary skill in the art to which the present invention belongs. Any method and material similar or equivalent to those described herein can be understood and used by those of ordinary skill in the art to practice the present invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims of the present invention are approximate and can vary depending upon the desired properties sought by the present invention.

The term “a/an” should mean one or more than one of the objects described in the present invention. The term “and/or” means either one or both of the alternatives. The term “a cell” or “the cell” may include a plurality of cells.

The term “and/or” is used to refer to both things or either one of the two mentioned.

The term “exosome” refers to cell-derived vesicles having a diameter of between about 20-140 nm, such as between 40 and 120 nm, preferably a diameter of about 50-100 nm, for example, a diameter of about 60 nm, 70 nm, 80 nm, 90 nm, or 100 mm. Exosomes may be isolated from any suitable biological sample from a mammal and cultured mammalian cells such as mesenchymal stem cells. As one of skill in the art will appreciate, cultured cell samples will be in the cell-appropriate culture media (using exosome-free serum). Exosomes include specific surface markers not present in other vesicles.

As used herein, the term “derived from” shall be taken to indicate that a particular sample or group of samples has originated from the species specified, but has not necessarily been obtained directly from the specified source.

The terms “treatment,” “treating,” and “treat” generally refer to obtaining a desired pharmacological and/or physiological effect. The effect maybe preventive in terms of completely or partially preventing a disease, disorder, or symptom thereof, and may be therapeutic in terms of a partial or complete cure for a disease, disorder, and/or symptoms attributed thereto. “Treatment” used herein covers any treatment of a disease in a mammal, preferably a human, and includes (1) suppressing development of a disease, disorder, or symptom thereof in a subject or (2) relieving or ameliorating the disease, disorder, or symptom thereof in a subject.

The terms “individual,” “subject,” and “patient” herein are used interchangeably and refer to any mammalian subject for whom diagnosis, treatment, or therapy is desired.

The term “pharmaceutical composition” of this invention includes an effective amount of the exosome, an exosome pellet, or physiological solution. The pharmaceutical composition of this disclosure is in liquid form, and it may contain pharmaceutically acceptable excipients that stabilize the liquid suspension and help cell viability.

The term “effective amount” refers to the amount of exosome, an exosome pellet, or physiological solution that, when administered to a patient or a subject in need for treating a disease or disorder, is sufficient to have a beneficial effect with respect to that disease or disorder. The therapeutically effective amount will vary depending on the conditions of the disease or disorder and its severity. It is not limited to the range stated in the specification. Determining the therapeutically effective amount of given exosome, an exosome pellet, or physiological solution is within the ordinary skill of the art and requires no more than routine experimentation.

The mesenchymal stem cells according to the disclosure can be obtained from different sources, preferably from umbilical cord, adipose tissue, or bone marrow. According to different sources, the mesenchymal stem cells are umbilical cord mesenchymal stem cells (UMSCs), adipose derived mesenchymal stem cells (ADSCs), and bone marrow mesenchymal stem cells (BMSCs). In some embodiments of this disclosure, MSCs are isolated and purified from the umbilical cord, and referred to as “umbilical MSC” or “UMSC.” In some embodiments, it is established that the UMSC in this disclosure expresses the same selection of surface markers as the MSC isolated from other bodies, and demonstrates comparable activities.

NASH or NAFLD is characterized by hyperinsulinemia, insulin resistance, hyperlipidemia, elevated serum transaminases such as aspartate aminotransferase (AST) and alanine aminotransferase (ALT) and liver cell injury driven by lipid accumulation, hepatic inflammation and lobular infiltration of inflammatory cells such as macrophages, and activation and transformation of hepatic stellate cells into smooth muscle cell phenotype. NASH frequently occurs in persons with conditions related to the metabolic syndrome such as obesity, diabetes mellitus, hyperlipidemia, high LDL cholesterol, low HDL cholesterol, and insulin resistance.

In another aspect, the present disclosure provides a method for treating non-alcoholic steatohepatitis or non-alcoholic fatty liver disease, reducing controlled attenuation parameter of liver fat, reducing hepatic lipid accumulation, decreasing hepatic lipotoxicity, ameliorating PPAR dysregulation in the liver, for reducing body weight, reducing glycated hematocrit, or improving insulin resistance in a subject in need thereof, wherein the method comprises administering the pharmaceutical composition as disclosed herein to the subject.

To date, PPAR's have been identified in the enhancers of a number of genes encoding proteins that regulate lipid metabolism suggesting that PPARs play a pivotal role in the adipogenic signaling cascade and lipid homeostasis. In some embodiments of the disclosure, the method is for ameliorating PPAR dysregulation in the liver. In some embodiments of the disclosure, the method is for decreasing PPARα and/or PPARγ expression.

A specific process is used to produce the exosome composition of the present disclosure. A desired amount of cells are provided firstly in a method for producing the exosome composition of the present disclosure. In some embodiments of the disclosure, the cells are cultured in a medium for a period of time sufficient to obtain a desired amount of cells. In some embodiments of the disclosure, the cell count is about 1×10⁵ cells/mL to about 1×10⁸ cells/mL; about 2×10⁵ cells/mL to about 8×10⁷ cells/mL; about 4×10⁵ cells/mL to about 6×10⁷ cells/mL; about 6×10⁵ cells/mL to about 4×10⁷ cells/mL; about 8×10⁵ cells/mL to about 2×10⁷ cells/mL; about 1×10⁶ cells/mL to about 1×10⁷ cells/mL; about 2×10⁶ cells/mL to about 8×10⁶ cells/mL; or about 4×10⁶ cells/mL to about 6×10⁶ cells/mL. The manners for culture depends on the cells. In some embodiments of the disclosure, the medium is a conditioned medium for obtaining the desired amount of cells with specific properties. For example, a conditioned medium is provided for maintaining cells at an undifferentiated stage or a differentiated stage.

The medium containing the desired amount of cells is subjected to a pre-clearance procedure for removing dead cells and/or cell debris. In one embodiment of the disclosure, the medium is centrifuged to remove dead cells. In some embodiments of the disclosure, dead cells can be removed at about 300 g, about 350 g, or about 400 g for about 5 minutes, about 6 minutes, about 7 minutes, about 8 minutes, about 9 minutes, about 10 minutes, about 11 minutes, about 12 minutes, about 13 minutes, about 14 minutes, or about 15 minutes. In one embodiment of the disclosure, the medium is centrifuged to remove cell debris. In some embodiments of the disclosure, cell debris can be removed at about 1500 g, about 1600 g, about 1700 g, about 1700 g, about 1800 g, about 1900 g, about 2000 g, about 2100 g, about 2200 g, about 2300 g, about 2400 g, or about 2500 g, for about 10 minutes, about 11 minutes, about 12 minutes, about 13 minutes, about 14 minutes, about 15 minutes, about 16 minutes, about 17 minutes, about 18 minutes, about 19 minutes, or about 20 minutes.

The resulting supernatant after removing the dead cells and/or cell debris is then filtrated to remove cell apoptotic bodies and microvesicles. In one embodiment of the disclosure, the resulting supernatant is filtrated by passing through about 0.15 about 0.16 about 0.18 about 0.20 about 0.22 about 0.24 about 0.26 or about 0.28 μm filter.

A type of membrane filtration in which forces (such as pressure or concentration gradients) lead to a separation through a semipermeable membrane. Ultrafiltration membranes are typically characterized by the molecular weight cut off of the membrane. Suspended solids and solutes of higher molecular weight are retained in the retentate, while water and lower molecular weight solutes pass through the membrane in the permeate. Different types of modules can be used for ultrafiltration processes. Examples of such modules are tubular elements that use polymeric membranes cast on the inside of plastic or paper tubes; hollow fiber designs that contain multiple hollow fibers; spiral wound modules in which flat membrane sheets are separated by a thin meshed spacer material that is rolled around a central perforated tube and fitted into a tubular steel pressure vessel casing; and plate and frame assemblies that use a membrane placed on a flat plate separated by a mesh like material through which the filtrate passes.

The resulting supernatant after ultrafiltration is then subjected to exosome precipitation for enrichment. In some embodiments of the disclosure, the resulting supernatant after ultrafiltration is precipitation with polymer-based precipitation. The resulting supernatant comprises a population of enriched exosomes in a concentration greater than about 1×10¹⁰ exosomes/mL.

In some embodiments of the disclosure, the exosomes are pelleted by centrifugation, e.g. at 10,000×g for 10 min at 4° C. to obtain the exosome pellet. In some embodiments of the disclosure, the exosome pellet is solubilized in a suitable saccharide solution, such as a trehalose solution, that is effective to reduce aggregation of the exosomes.

This disclosure provides methods for treating fatty liver disease which may include but are not limited to secondary NAFLD, steatosis, progressive fibrosis, liver failure and cirrhosis. As used herein, secondary NAFLD may refer to NAFLD or similar symptoms that result from the use of one or more of the following medications: amiodarone, antiviral drugs such as nucleoside analogues, aspirin or NSAIDs, corticosteroids, methotrexate, nifedipine, perhexyline, tamoxifen, tetracycline, and valproic acid.

Certain embodiments of the effective amount of the exosomes of the present disclosure are those ranging from about 1×10⁵ particles to about 1×10¹⁵ particles. In some embodiments, the effective amount of the exosomes of the present disclosure are those ranging from about 1×10⁶ particles to about 1×10¹⁴ particles, from about 1×10⁷ particles to about 1×10¹′ particles, from about 1×10⁸ particles to about 1×10¹² particles, from about 1×10⁸ particles to about 1×10¹¹ particles, from about 1×10⁸ particles to about 1×10¹⁰ particles, from about 5×10⁸ particles to about 5×10⁹ particles, about 1×10⁹ particles.

The pharmaceutical compositions disclosed herein can be administered by one of many routes, depending on the embodiment. For example, exosome administration may be by local or systemic administration. Local administration, may in some embodiments be achieved by direct administration to a tissue (e.g., direct injection, such as intramyocardial injection). Local administration may also be achieved by, for example, lavage of a particular tissue (e.g., intra-intestinal or peritoneal lavage). In several embodiments, systemic administration is used and may be achieved by, for example, intravenous and/or intra-arterial delivery. In certain embodiments, intracoronary delivery is used. In several embodiments, the exosomes are specifically targeted to the damaged or diseased tissues. In some such embodiments, the exosomes are modified (e.g., genetically or otherwise) to direct them to a specific target site.

The administration of the pharmaceutical composition may be performed more than one time; preferably, the pharmaceutical composition is administrated to the subject twice in a time interval. The interval between the administrations may be 1, 2, 3, 4, 5, 6, 7, 8, 9, 10, 11, 12, 13, or 14 days, preferably, 7 days. In some embodiments of the disclosure, the exosome, exosome pellet, or physiological solution is administered every other day.

It is to be understood that if any prior art publication is referred to herein, such reference does not constitute an admission that the publication forms a part of the common general knowledge in the art.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### EXAMPLES

Isolation of Exosomes Derived from Mesenchymal Stem Cells

Collection of conditional medium: Mesenchymal stem cells were incubated at a seeding density of 5000/cm² for two days. After being washed once with PBS, the MSCs culture was supplemented with serum-free medium or medium containing exosomal serum and further incubated for 48 hours at 37° C. in an incubator. The conditioned medium was collected.

Pre-treatment of the conditional medium: Dead cell debris was removed from the conditional medium by centrifugation at 350 g for 10 minutes and at 2000 g for 15 minutes (4° C.). The apoptotic vesicles contained in the conditioned medium were removed by filtering through a 0.22 μm pore size.

Concentration of conditional medium: The conditional medium was concentrated by centrifugation at 4° C. using protein concentration ultrafiltration to concentrate 20 ml of conditional medium to 1 ml.

Isolation and extraction of exosomes: The concentrated conditioned medium was mixed with ExoPEG (polymer-based precipitating) in equal volume and incubated at 4° C. for 1 hour or overnight. The supernatant was removed by centrifugation at 10,000 g for 30 minutes and at 2000 g for 5 minutes (4° C.) and further removed the supernatant residue. The pellet was mixed with 200 μL of PBS and placed on ice for 20 minutes for suspending and dispersing, and then stored quickly at −80° C.

Quantification of exosomes: The prepared mesenchymal stem cell exosomes were quantified by BCA (bicinchoninic acid) protein analysis.

### Example 1 Results of Exosomes Derived from Mesenchymal Stem Cells for Treating Non-Alcoholic Steatohepatitis

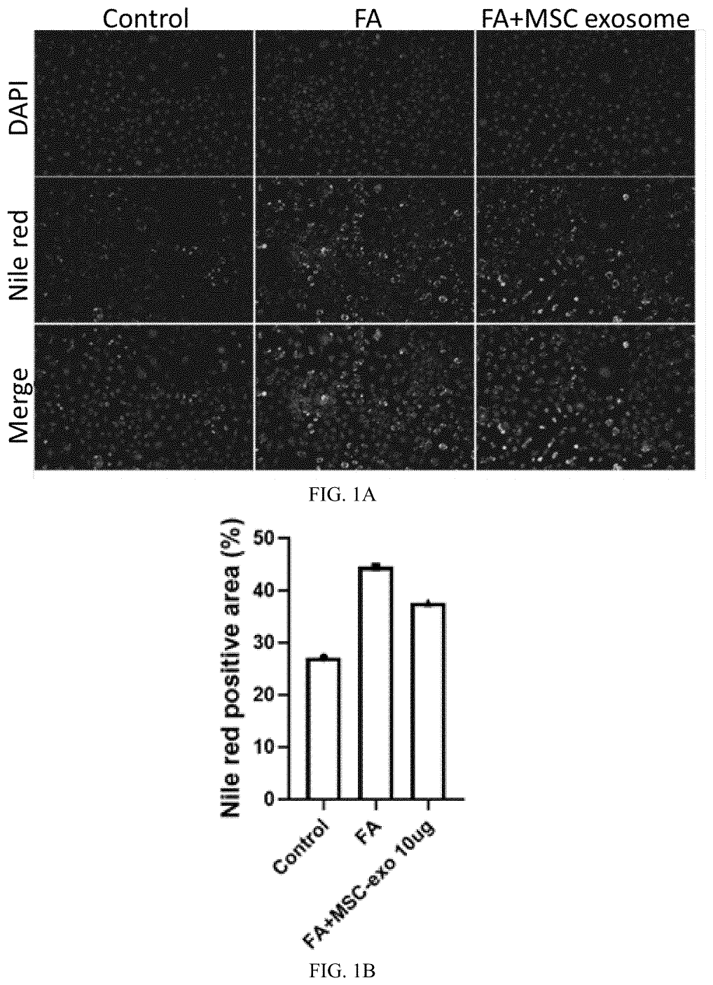

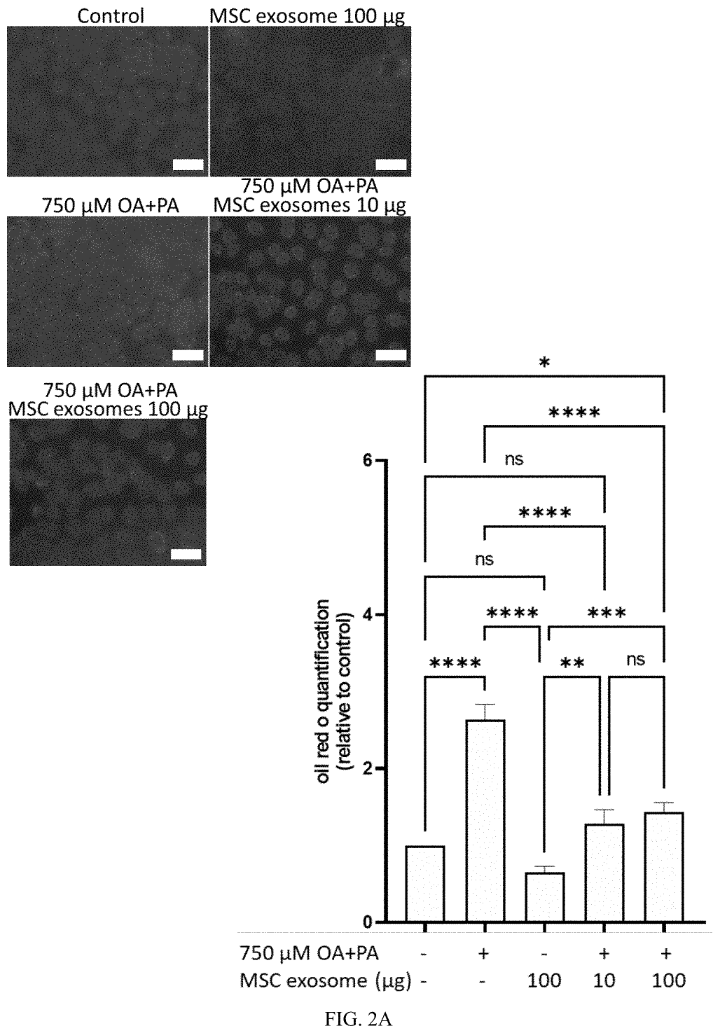

FL83B was cultured with DMEM/F12 (Thermo Fisher Scientific, Waltham, Mass., USA) with 10% FBS (Thermo Fisher Scientific, Waltham, Mass., USA) and 1% PSG (Penicillin-Streptomycin-Glutamine; Thermo Fisher Scientific, Waltham, Mass., USA) at 37° C., 5% CO₂. The cells were pretreated with 200 μM of free fatty acids (FA, palmitic acid to oleic acid with 2:1) for 24 hours for inducing lipid accumulation. The pretreated cells were treated with exosomes derived from MSCs (10 μg per 6-well) for one day. Nile red staining was used for assaying lipid accumulation in FL83B. Under observation with the fluorescence microscope as shown in FIGS. 1A and 1B, exosomes derived from mesenchymal stem cells could reduce the accumulation of lipid particles.

### Example 2 Exosomes from Human MSCs Decrease Fatty Acid-Induced Intracellular Lipid Accumulation and Lipotoxicity in a Hepatoblast Cell Line

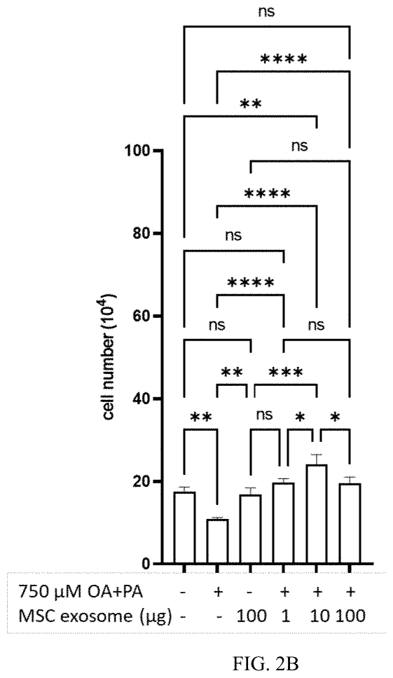

FL83B cells were seeded in 6-well plates and were treated with free fatty acids 750 μM (palmitic acid to oleic acid with 2:1) for two days and then treated with exosomes from human MSCs (1-100 μg per 6-well) for another two days. Under observation with the fluorescence microscope as shown in FIGS. 2A and 2B, exosomes derived from mesenchymal stem cells decrease fatty acid-induced intracellular lipid accumulation and lipotoxicity in a hepatoblast cell line, FL83B cells.

### Example 3 Exosomes from Human MSCs Ameliorate PPAR Dysregulation in a Hepatoblast Cell Line

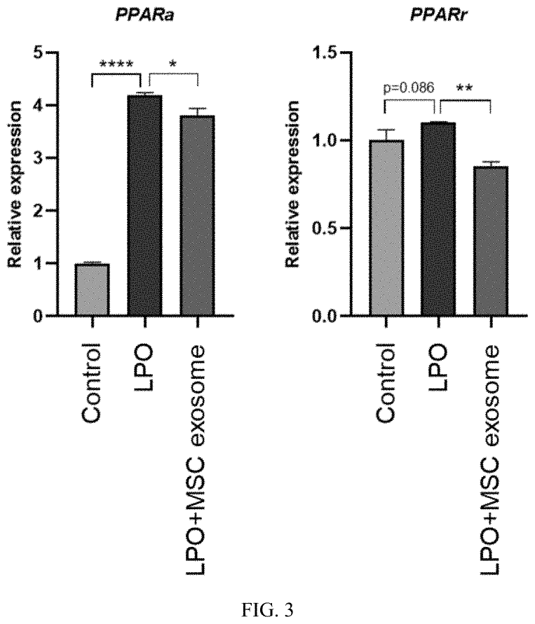

FL83B cells were exposed to LPO medium to mimic hepatic steatosis and treated with or without exosomes (2.5×10⁹) from MSCs. Real-time qPCR revealed the expression of PPARα and PPARγ, the master regulators of lipid metabolism, in FL83B cells. LPO medium, DMEM/F12 medium+10% FBS+1% PSG+10 mM L-lactate+1 mM sodium pyruvate+4 mM octanoic acid. As shown in FIG. 3, exosomes derived from mesenchymal stem cells decrease PPARα and PPARγ expression in a hepatoblast cell line, FL83B cells.

### Example 4 Exosome from Human MSCs Reduced Body Weight in HFD Mice

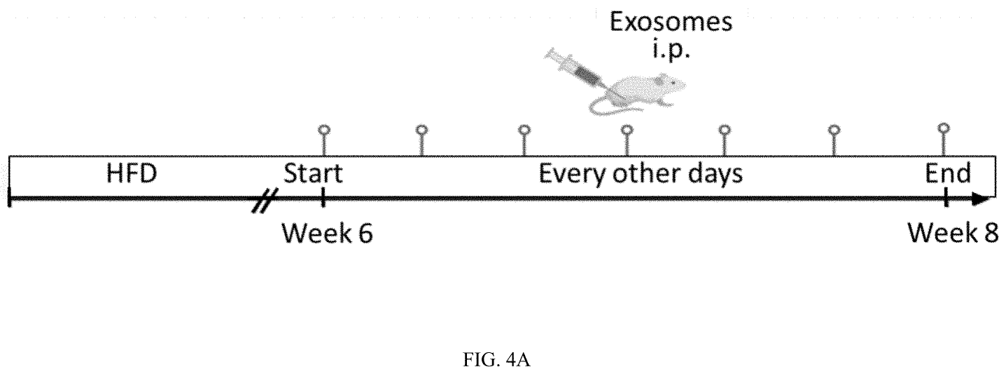

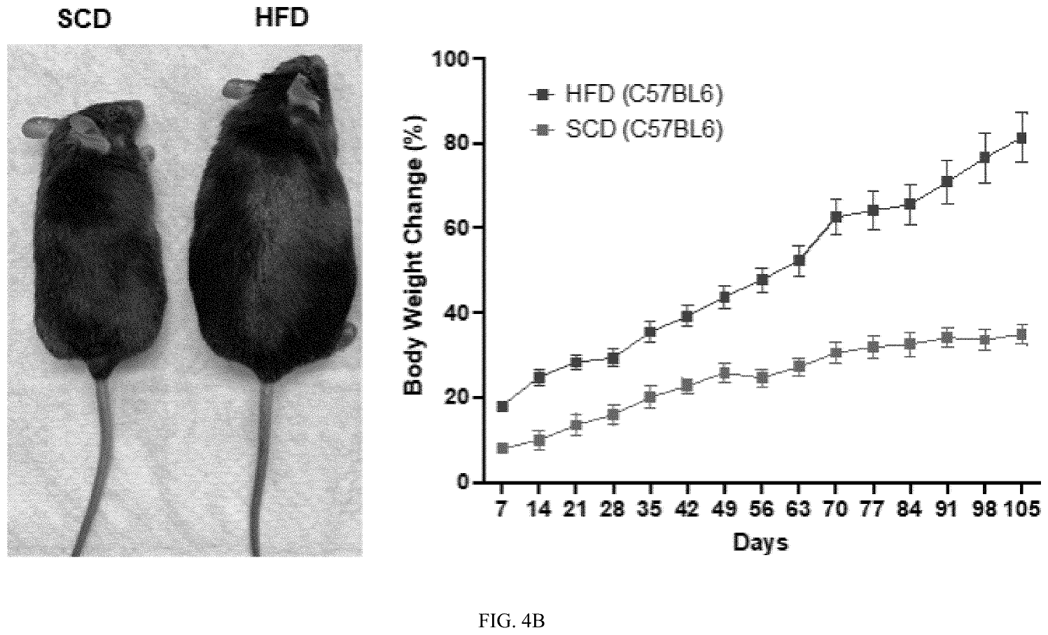

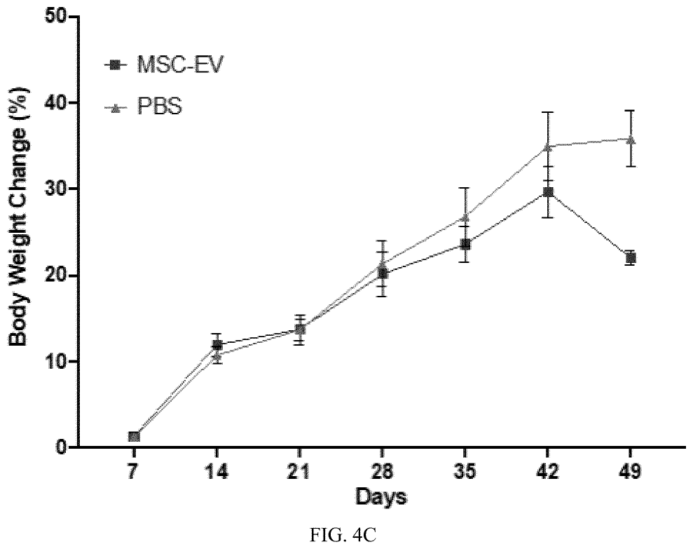

FIG. 4A shows experimental setup. Mice were exposed to either the SCD or HFD for sixteen weeks. FIG. 4B shows that HFD increased body weight in mice. Eight animals per group were individually analyzed. Obese mice were randomly divided into two groups (normal and treatment, n=8/group), and the treatment of MSC-derived exosomes was performed for 15 days (0.5 mg/kg/every other day). FIG. 4C shows that exosome injection (10⁹ particle per injection) reduced body weight in HFD mice.

While the present disclosure has been described in conjunction with the specific embodiments set forth, many alternatives thereto and modifications and variations thereof will be apparent to those of ordinary skill in the art. All such alternatives, modifications and variations are considered to fall within the scope of the present disclosure.

What is claimed is:

1. A method for treating non-alcoholic steatohepatitis and/or non-alcoholic fatty liver disease (NAFLD) in a subject in need thereof, wherein the method comprises administering an exosome, exosome pellet, or physiological solution derived from mesenchymal stem cells (MSCs) to the subject.

2. The method of claim 1, wherein the method is for reducing hepatic lipid accumulation.

3. The method of claim 1, wherein the method is for decreasing hepatic lipotoxicity.

4. The method of claim 1, wherein the method is for ameliorating PPAR dysregulation in the liver.

5. The method of claim 1, wherein the method is for decreasing PPARα and/or PPARγ expression.

6. The method of claim 1, wherein the method is for reducing body weight.

7. The method of claim 1, wherein the method is for reducing body weight in a subject receiving high-fat diets.

8. The method of claim 1, wherein the method is for stimulating serum alanine aminotransferase expression.

9. The method of claim 1, wherein the method is for reducing controlled attenuation parameter of liver fat.

10. The method of claim 1, wherein the method is for reducing glycated hematocrit.

11. The method of claim 1, wherein the method is further for improving insulin resistance.

12. The method of claim 1, wherein the NAFLD is secondary NAFLD, steatosis, progressive fibrosis, liver failure or cirrhosis.

13. The method of claim 12, wherein the secondary NAFLD is an NAFLD resulted from the use of one or more of amiodarone, antiviral drugs such as nucleoside analogues, aspirin or NSAID s, corticosteroids, methotrexate, nifedipine, perhexyline, tamoxifen, tetracycline, or valproic acid.

14. The method of claim 1, wherein the exosome, exosome pellet, or physiological solution is administered by injection.

15. The method of claim 1, wherein the exosome, exosome pellet, or physiological solution is administered more than one time.

16. The method of claim 1, wherein the exosome, exosome pellet, or physiological solution is administered to the subject twice in a time interval.

17. The method of claim 1, wherein the exosome, exosome pellet, or physiological solution is administered every other day.

18. The method of claim 1, wherein the MSCs are umbilical cord mesenchymal stem cells (UMSCs), adipose derived mesenchymal stem cells (ADSCs), or bone marrow mesenchymal stem cells (BMSCs).

19. The method of claim 1, wherein the exosome, exosome pellet, or physiological solution is obtained by culturing cells in a medium for a period of time sufficient to obtain an amount of cells ranging from about 1×10⁵ cells/mL to about 1×10¹⁰ cells/mL.

20. The method of claim 1, wherein the amount of the exosome ranges from about 1×10⁵ particles to about 1×10¹⁵ particles.
